(19) United States
(12) Patent Application Publication
(10) Pub. No.: US 2006/0292642 A1
(43) Pub. Date: Dec. 28, 2006

(54) Immunoassay of phosphorylated proteins

(76) Inventors: Javad Khosravi, Toronto (CA); Gopal V. Savjani, Nassau Bay, TX (US)

Correspondence Address: BECKMAN COULTER, INC., P.O. BOX 169015, MAIL CODE 32-A02, MIAMI, FL, 33116-9015, US

(21) Appl. No.: 11/473,909

(22) Filed: Jun. 23, 2006

Related U.S. Application Data
(60) Provisional application No. 60/694,105, filed on Jun. 24, 2005.

(51) Int. Cl. G01N 33/542; G01N 33/53

(52) U.S. Cl. 435/7.9

(57) ABSTRACT

An immunoassay of phosphorylated isoforms (phosphoforms, phosphoisoforms) of peptides, proteins, or their various related forms is described. In one embodiment, a sandwich-type “two-site” immunoassays involving two different recognition antibody partners, in which one antibody is specific for the protein or peptide and the other is specific for a known or discovered phosphorylated amino acid residue. An assay embodiment involves a first-step capturing of the protein or peptide with a specific anti-protein antibody, and a second-step detection of the bound phosphorylated isoform of the protein with an antibody directed against a known phosphorylated residue coupled to a label or a reporter molecule. The various embodiments of the compositions and methods of the invention are exemplified by immunoassays for phosphoforms of IGFBPs, such as IGFBP-1 and IGFBP-5, which contain phosphorylated serine residues.

### CROSS REFERENCE TO RELATED APPLICATIONS

This application claims the benefit of the priority of U.S. provisional patent application No. 60/694,105, filed Jun. 24, 2005.

### BACKGROUND OF THE INVENTION

Insulin-like growth factors (IGF-I and IGF-II) belong to a family of peptides that mediate a broad spectrum of growth hormone-dependent and independent mitogenic and metabolic actions essential for cell growth and development (1-4). Unlike most peptide hormones, IGFs in circulation and in other physiological fluids are associated with a group of high-affinity Insulin-like growth factor binding proteins (IGFBPs) that specifically bind and modulate IGF bioactivity at the cellular level. Six structurally homologous IGFBPs with distinct molecular size, hormonal control, tissue expression and functions have been identified (4-6).

IGFBP-1, synonymous with placental protein-12 (7) and the pregnancy-associated endometrial α₁-globulin (8), is a 25 kilodalton (kDa) protein expressed and secreted by a variety of cell types, including hepatocytes, ovarian granulosa cells, and decidualized endometrium (9-11). IGFBP-1 is present in serum, is the predominant IGF binding protein in amniotic fluid, and is the major IGF binding protein in fetal and maternal circulation (9, 12-13). In both humans and animal models, elevated levels of IGFBP-1 have been found in association with fetal growth restrictions (9, 13-17).

IGFBP-1 is reportedly capable of both inhibition as well as augmentation of IGF action (4, 6). These dual functionalities of IGFBP-1 have been partly explained by posttranslational phosphorylation of amino acid residues. Posttranslational phosphorylation of a serine amino acid residue alters the affinity of IGFBP-1 for the IGFs by four to eight fold (4, 18), thereby affecting its capacity to regulate IGF bioavailability. Up to five IGFBP-1 variants have been identified, differing only in their degree of phosphorylation. Various cell types such as Hep G2, decidual cells, and liver cells have been found to secrete predominantly phosphorylated forms of IGFBP-1, whereas amniotic fluid and fetal serum contain substantial amounts of non-phosphorylated IGFBP-1 variants (4, 18-19). Although a predominantly phosphorylated form of IGFBP-1 is found in normal adult sera, the phosphorylation profile of IGFBP-1 is subject to significant changes in response to pregnancy (20).

Post-translational modification of amino acid residues, such as reversible phosphorylation, is an essential and almost universal mechanism of protein activation and deactivation, and is responsible for regulating nearly all cell signaling pathways and ultimately all biological functions (21). Significant progress in the identification of various phosphorylated proteins (“phosphoproteins” or “phosphoforms”) and the localization of phosphorylated residues has been achieved by various methodologies, including mass spectrometry (22). Accordingly, regulated phosphorylation/dephosphorylation of IGFBPs has been also recognized as an important alternative mechanism by which bioavailability of the IGFs might be modulated (4, 23). In addition to normal pregnancy (20), studies of IGFBP-1 have so far identified significant changes in the phosphorylation profiles of IGFBP-1 in subjects with Larone syndrome, (20, 24) as well as in relation to pre-eclampsia (25), postnatal growth restriction (15), regulation of biosynthesis of collagen and glycosaminoglycan (25), and in relation to the development of cardiovascular disease and vascular complications of type 2 diabetes (26). Research interest has been more recently extended to studies of other members of the IGFBP family, such as IGFBP-3 and IGFBP-5, which are also known to be serine-phosphorylated (4, 23). Similar to IGFBP-1, the available evidence appears to support a significant role for altered phosphorylation of IGFBP-3 and IGFBP-5 in regulating their various binding characteristics and IGF-dependent and/or IGF-independent functions (4, 27-29). IGFBP-5 has been more extensively studied in relation to bone metabolism and osteoporosis (4, 6, 29, 45).

Studies of the mechanism of protein activation and/or deactivation by phosphorylation suggest that the effect of phosphorylation is mediated by inducing important conformational changes within the ternary structure of the proteins (30). As the immunological basis of antigen/antibody interactions is also largely dependent on recognition of conformational epitopes (31), investigations of the effect of altered phosphorylation in relation to potential changes in immunoreactivity are of significant importance (21, 30). Although traditional immunoassays with broad specificity for proteins have contributed to such investigations, the availability of simple immunoassays for the specific determination of phosphoforms of various proteins would expedite the understanding of their regulatory mechanisms, pathophysiological roles, and clinical relevance.

In the field of IGF research, a significant relationship between protein phosphorylation and immunoreactivity was first described for IGFBP-1, where as a result of differential recognition of IGFBP-1 phosphoforms by different antibodies, up to ten-fold differences in the measured concentrations of IGFBP-1 in normal adult sera were observed (20, 32). Variable recognition of IGFBP-1 phosphoforms by antibodies may result in false estimates or in inappropriate interpretations of the measured IGFBP-1 levels. Significant changes in immunoreactivity of IGFBP-5 in response to altered phosphorylation have also been observed. The latter involved a systematic evaluation of a panel of four polyclonal and 12 monoclonal antibodies raised against recombinant human IGFBP-5 or a synthetic IGFBP-5 peptide. IGFBP-5 immunoreactivity in both competitive (EIA) and non-competitive (ELISA) formats was found to be significantly affected by Mg²⁺, EDTA, and the phosphorylation status of the IGFBP-5 molecule (33, 34).

Studies of phosphorylated proteins by the conventional immunoassays may be affected by effects of altered phosphorylation on the levels of the detectable immunoreactivity. As described for IGFBP-1 and IGFBP-5, altered phosphorylation may result in variations in the detectable serum levels. This is particularly important considering the variable expression of IGFBP-1 phosphorylated isoforms observed in biological fluids (4, 18, 19, 32). The validity of the reported IGFBP-1 normal ranges, even in the nonpregnant adult population, which apparently expresses a single IGFBP-1 variant, has been questioned (20). In addition, IGFBP-1 antibodies have been reported to detect significantly different serum concentrations in nonpregnant subjects (up to 11-fold differences in the mean values), while measuring relatively similar concentrations during pregnancy (20). Immunoassays for IGFBPs have been developed where the antibody immunoreactivity is unaffected by phosphorylation of the protein (32, and U.S. Pat. No. 5,747,273). Such an immunoassay allows the detection of the total concentration of the IGFBP in a sample regardless of the degree of phosphorylation. However, such an immunoassay cannot measure the level of protein phosphorylation or detect changes in protein phosphorylation levels.

Although detection of various protein phosphoforms has been possible by the western and/or ligand immunoblots after gel electrophoretic separation of molecules of interest (18, 20), these methods involve limitations in the number of samples that can be assayed per run, labor intensiveness, high cost, and particularly the qualitative or at best semi-quantitative nature of the analyses.

Given the ever increasing importance of protein phosphorylation, there remains an urgent need for simple methodology allowing for the quantification of large numbers of samples (21, 22). Although changes in the phosphorylation level of IGFBPs are documented by traditional approaches, such as sodium dodecyl sulfate polyacrylamide gel electrophoresis (SDS-PAGE) and ligand and/or immunoblotting approaches (4, 18-20), the available methodologies are at best semi-quantitative and not suitable for large scale manual or automated applications.

There remains a need in the art for comparatively simple and reliable quantitative immunoassay methodologies and compositions that permit the measurement of phosphorylated protein variants and provide for large scale sample analysis by manual and/or fully automated operations.

### SUMMARY OF THE INVENTION

Methods and compositions relating to immunoassays of phosphorylated isoforms of proteins or peptides are disclosed herein. More specifically, the immunoassays described herein relate to phosphorylated isoforms of Insulin-like growth factor binding proteins (IGFBP).

Various embodiments of methods and compositions described herein provide the ability to quantify phosphorylated isoforms of proteins, such as IGFBP, using the advantages and simplicity of the conventional immunoassay format. Combining an anti-IGFBP capture antibody with an anti-phosphorylated residue antibody represents a novel approach to immunoassay of phosphorylated proteins, as exemplified here for IGFBPs.

One embodiment of an immunoassay composition described herein comprises a first antibody and a second antibody. The first antibody binds to a protein having a phosphorylated amino acid residue, but does not bind to the phosphorylated amino acid residue. The second antibody binds to the phosphorylated amino acid residue.

An additional embodiment includes an immunoassay kit for measuring a concentration of a protein having a phosphorylated amino acid residue in a sample is also described herein. In one embodiment, such a kit comprises a first antibody and a second antibody, wherein the first antibody binds to a protein having a phosphorylated amino acid residue and the second antibody binds to the phosphorylated amino acid residue. According to this embodiment, the kit also contains a solid support coupled with the first antibody and a label coupled with the second antibody.

In another aspect, an immunoassay method is described herein for measuring a concentration of a protein having a phosphorylated amino acid residue in a sample. In one embodiment, a method comprises binding a first antibody to a protein having a phosphorylated amino acid residue, thereby creating a bound first antibody. A second antibody is bound to the phosphorylated amino acid residue, thereby creating a bound second antibody. The amount of the bound second antibody is measured; and the concentration of the protein in the sample is calculated based on the amount of bound second antibody.

Yet an additional embodiment is an immunoassay method for measuring a phosphorylation level of a protein sample. This method comprises the steps of: contacting a first antibody with a protein sample, wherein the protein sample comprises a protein having a phosphorylated amino acid residue, and wherein the first antibody binds to the protein, thereby creating abound first antibody. The method also includes the step of binding a second antibody to the phosphorylated amino acid residue, thereby creating a bound second antibody. An amount of bound second antibody is measured, and a concentration of the protein having a phosphorylated amino acid residue in the sample is calculated based on the amount of bound second antibody. A concentration of total protein in the protein sample is calculated. The concentration of the protein having a phosphorylated amino acid residue is then determined relative to the concentration of total protein in the sample.

Other and further aspects, features, and advantages of the present invention are apparent from the following description of the presently preferred embodiments of the invention. These embodiments are provided for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the various embodiments of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 is a graph showing the concentration (μg/L) of phosphorylated IGFBP-1 in serum samples measured by enzyme-linked immunosorbent assay (ELISA), absorbance at 450 nm. A pre-selected human serum pool was assayed under three conditions. One group of untreated serum samples was serially diluted and assayed using an anti-phosphoserine detection antibody (●). Another group of untreated serum samples was serially diluted and assayed using an anti-phosphotyrosine detection antibody (∘). A third group of serum samples was dephosphorylated by treatment with alkaline phosphatase (ALP), then serially diluted, and assayed using the anti-phosphoserine detection antibody (▾). Values shown are the means of duplicate measurements. See Example 1.

FIG. 2 is a graph showing the concentration of phosphorylated IGFBP-5 (units/mL) by ELISA, absorbance at 450 nm. A pre-selected human serum pool was serially diluted and assayed using the anti-phosphoserine detection antibody (●). The serial dilutions of the pool were also assayed using the anti-phosphotyrosine antibody (O). Values shown are the means of duplicate measurements. See Example 1.

FIG. 3 is a bar graph showing the specificity of an ELISA measuring phosphorylated IGFBP-5 in human serum samples (n=16), in absorbance at 450 nm. IGFBP-5 captured by anti-IGFBP-5 polyclonal antibody #1 was detected by either anti-phosphoserine antibody (col. 1) or anti-phosphotyrosine antibody (col. 2). IGFBP-5 captured by anti-IGFBP-5 monoclonal antibody #4 was detected by either anti-phosphoserine antibody (col. 4) or anti-phosphotyrosine antibody (col. 5). IGFBP-5 was captured by either anti-IGFBP-5 monoclonal antibody #3 (col. 3) or anti-IGFBP-5 polyclonal antibody #6 (col. 6) and detected by anti-phosphoserine antibody. The median levels of IGFBP-5 measured and 95% confidence intervals are plotted. See Example 1.

FIG. 4 is a bar graph showing the specificity of an ELISA for phosphorylated IGFBP-5 ELISA in human serum samples (n=16). Col. 1 represents samples assayed for phosphorylated IGFBP-5 before dephosphorylation by alkaline phosphatase (ALP), using capture polyclonal antibody #1 and the detection anti-phosphoserine antibody. Col. 2 represents samples assayed for phosphorylated IGFBP-5 after dephosphorylation by ALP, using capture polyclonal antibody #1 and the detection anti-phosphoserine antibody. Col. 3 represents samples assayed for phosphorylated IGFBP-5 before dephosphorylation by ALP, using capture monoclonal antibody #3 and the detection anti-phosphoserine antibody. Col. 4 represents samples assayed for phosphorylated IGFBP-5 after dephosphorylation by ALP, using capture monoclonal antibody #3 and the detection anti-phosphoserine antibody. The median levels of IGFBP-5 measured and 95% confidence intervals are plotted. See Example 1.

FIGS. 5A-5C show the concentration of various forms of IGFBP-1 (μg/L) measured by ELISA in non-pregnant human serum (n=29) (column 1), in human sera from a first trimester pregnancy (n=38) (column 2), in human sera from a second trimester pregnancy (n=29) (column 3) and in human amniotic fluids (n=20) (column 4). The median levels of IGFBP-1 measured and the 95% confidence intervals are shown. FIG. 5A is a bar graph showing the concentration of total IGFBP-1, including both phosphorylated and non-phosphorylated IGFBP-1 in the four fluid samples identified above. FIG. 5B is a bar graph showing the concentration of non-phosphorylated IGFBP-1 only in the same samples. FIG. 5C is a bar graph showing the concentration of phosphorylated IGFBP-1 only in the same samples. See Example 2.

FIG. 6 is a bar graph showing ratios of median levels of total IGFBP-1 (filled bars), the non-phosphorylated IGFBP-1 variants only (unfilled, grey bars) and phosphorylated IGFBP-1 variants only (cross-hatched bars) compared to the total measured IGFBP-1. IGFBP-1 concentrations were measured by ELISA in human non-pregnancy serum samples (bars 1-3), first trimester serum samples (bars 4-6), second trimester serum samples (bars 7-9), and amniotic fluid samples (bars 10-12). See Example 2.

FIG. 7A is a graph showing an analysis of phosphorylated IGFBP-1 in non-pregnant human serum samples versus total IGFBP-1 immunoreactivity measured by ELISA. (PO4)-IGFBP-1 in μg/L is plotted on the Y axis. Total IGFBP-1 in 1 g/L is plotted along the X axis. Values plotted are the means of duplicate measurements. Plotting and statistical analysis parameters were y=0.88x−2.27; r=0.99; Sy/x=4.5; and p=<0.001. See Example 2.

FIG. 7B is a graph showing a comparative analysis similar to that of FIG. 7A of phosphorylated IGFBP-1 in first trimester pregnant human serum samples vs total IGFBP-1 immunoreactivity measured by ELISA. Plotting and statistical analysis parameters were y=0.41x+3.19; r=0.48; Sy/x=32.5; and p=0.002. See Example 2.

FIG. 7C is a graph showing a comparative analysis similar to that of FIG. 7A of phosphorylated IGFBP-1 in second trimester pregnant human serum samples vs. total IGFBP-1 immunoreactivity measured by ELISA. Plotting and statistical analysis parameters were y=0.49x−1.09; r=0.74; Sy/x=20; and p=<0.001. See Example 2.

FIG. 8A is a graph showing an analysis of phosphorylated IGFBP-1 in non-pregnant human serum samples vs. non-phosphorylated IGFBP-1 immunoreactivity measured by ELISA. (PO4)-IGFBP-1 in μg/L is plotted on the Y axis. Non-phosphorylated IGFBP-1 in μg/L is plotted along the X axis. Values plotted are the means of duplicate measurements. Plotting and statistical analysis parameters were y=9.08x+4.7; r=0.93; Sy/x=12; and p=<0.001. See Example 2.

FIG. 8B is a graph showing a comparative analysis similar to that of FIG. 8A of phosphorylated IGFBP-1 in first trimester pregnant human serum samples vs non-phosphorylated IGFBP-1 immunoreactivity measured by ELISA. Plotting and statistical analysis parameters were y=1.29x+7.9; r=0.38; Sy/x=32; and p=0.018. See Example 2.

FIG. 8C is a graph showing a comparative analysis similar to that of FIG. 8A of phosphorylated IGFBP-1 in second trimester pregnant human serum samples vs. non-phosphorylated IGFBP-1 immunoreactivity measured by ELISA. Plotting and statistical analysis parameters were y=0.74x+18.15; r=0.46; Sy/x=27; and p=0.003. See Example 2.

### DETAILED DESCRIPTION OF THE INVENTION

I. Compositions of the Invention

An immunoassay composition, as described herein, comprises a first antibody and a second antibody. The first antibody binds to a protein having a phosphorylated amino acid residue, but does not bind to the phosphorylated amino acid residue. The second antibody binds to the phosphorylated amino acid residue. In one embodiment, the composition is an intermediate provided by the first antibody bound to the “target” protein, which is bound at its phosphorylated amino acid residue to the second antibody. As described in detail below, the first antibody is optionally bound to a solid support. In another embodiment, the second antibody is optionally bound to a label. In another embodiment, the first antibody is bound to the target protein at a first epitope; and the binding of the first antibody to the first epitope does not interfere with the binding of the second antibody to the phosphorylated residue. In another embodiment, the first epitope differs from the second epitope, which contains the phosphorylated residue or is the phosphorylated residue. Another embodiment of a composition of this invention is a product or collection of the individual components that make up the composition.

Thus, another embodiment of such a composition is an immunoassay kit for measuring a concentration of a protein having a phosphorylated amino acid residue in a sample. In one embodiment, such a kit comprises a first antibody and a second antibody, wherein the first antibody binds to a protein having a phosphorylated amino acid residue, but does not bind to the phosphorylated amino acid residue, and the second antibody binds to the phosphorylated amino acid residue. According to this embodiment, the kit also contains a solid support coupled with the first antibody and a label coupled with the second antibody. Suitable examples of solid supports are identified below. Suitable examples of labels for use in the kit are similarly identified below. The kit also contains optional additional components for performing assays methods described herein. Such optional components are independently selected from containers, mixers, instructions for assay performance, labels, supports, and reagents necessary to couple the antibody to the support or label

Descriptions of the components of these compositions, products and kits including the antibodies, target protein, supports, labels and optional kit components are provided in more detail below.

II. Methods of the Invention

An embodiment of a method of the invention is an immunoassay method for measuring a concentration of a protein having a phosphorylated amino acid residue in a sample, comprising the steps of binding a first antibody to a protein having a phosphorylated amino acid residue, thereby creating a bound first antibody; binding a second antibody to the phosphorylated amino acid residue, thereby creating a bound second antibody; measuring an amount of the bound second antibody; and calculating the concentration of the protein in the sample based on the amount of bound second antibody.

In one embodiment, a one-step assay (simultaneous incubation of sample plus detection antibody) is useful. In another embodiment, a two-step assay (sequential incubation of sample and the detection antibody) is useful. A two-step assay is preferable in the case where other phosphorylated molecules could compete for binding to the anti-phosphorylated moiety e.g., anti-phosphoserine or phosphotyrosine.

In an embodiment of an immunoassay referred to as immunometric, “two-site” or “sandwich” immunoassay, the analyte is bound to or sandwiched between two antibodies that bind to different epitopes on the analyte. Representative examples of such immunoassays include enzyme immunoassays or enzyme-linked immunosorbent assays (EIA or ELISA), immunoradiometric assays (IRMA), fluorescent immunoassays, lateral flow assays, diffusion immunoassays, immunoprecipitation assays, and magnetic separation assays (MSA). In one such assay, a first antibody, which is described as the “capture” antibody, is bound to a solid support, such as a protein coupling or protein binding surface, colloidal metal particles, iron oxide particles, or polymeric beads. One example of a polymeric bead is a latex particle. In such an embodiment, the capture antibody is bound to or coated on a solid support using procedures known in the art. Alternatively, the capture antibody is coupled with a ligand that is recognized by an additional antibody that is bound to or coated on a solid support. Binding of the capture antibody to the additional antibody via the ligand then indirectly immobilizes the capture antibody on the solid support. An example of such a ligand is fluorescein.

The second antibody, which is described as the “detection” antibody, is coupled or conjugated with a label using procedures known in the art. Examples of suitable labels for this purpose include a chemiluminescent agent, a colorimetric agent, an energy transfer agent, an enzyme, a substrate of an enzymatic reaction, a fluorescent agent and a radioisotope. In one embodiment, the label includes a first protein such as biotin coupled with the second antibody, and a second protein such as streptavidin that is coupled with an enzyme. The second protein binds to the first protein. The enzyme produces a detectable signal when provided with substrate(s), so that the amount of signal measured corresponds to the amount of second antibody that is bound to the analyte. Examples of enzymes include, without limitation, alkaline phosphatase, amylase, luciferase, catalase, beta-galactosidase, glucose oxidase, glucose-6-phosphate dehydrogenase, hexokinase, horseradish peroxidase, lactamase, urease and malate dehydrogenase. Suitable substrates include, without limitation, TMB (3,3′,5,5′-tetramethyl benzidine, OPD (o-phenylene diamine), and ABTS (2,2′-azino-bis(3-ethylbenzthiazoline-6-sulfonic acid).

An additional embodiment of an immunoassay method is designed for measuring phosphorylation level of a protein sample. Such a method comprises the steps of contacting a first antibody with a protein sample, wherein the protein sample comprises a target protein having a phosphorylated amino acid residue. The first antibody binds to the protein, thereby creating a bound first antibody. A second antibody is contacted with the sample, and binds to the phosphorylated amino acid residue of the target protein, thereby creating a bound second antibody. The amount of bound second antibody is measured. The concentration of the protein having a phosphorylated amino acid residue in the sample is calculated based on the amount of bound second antibody. The concentration of total target protein in the protein sample is determined, and the concentration of the target protein having a phosphorylated amino acid residue relative to the concentration of total protein in the sample is determined. In one embodiment of such an immunoassay method, relating the concentration of the protein having a phosphorylated amino acid residue to the concentration of total protein in the sample involves calculating a ratio of the concentration of the protein having a phosphorylated amino acid residue and the concentration of total protein in the sample.

The various embodiments of the described compositions and methods are used to measure phosphorylated variants or phosphoforms of any protein having a phosphorylated amino acid residue. In embodiments herein, the first antibody that binds to a phosphorylated protein binds to a first epitope in the protein, and the second antibody binds to a second epitope in the protein that contains a phosphorylated amino acid residue. In one embodiment, the epitope bound by the second antibody comprises the phosphorylated amino acid residue and optionally other amino acid residues flanking it. In another embodiment, the epitope bound by the second antibody is a single phosphorylated amino acid residue. The phosphorylated amino acid residue to which the second antibody binds is any of the amino acid residues that are phosphorylated in proteins, including, without limitation, phosphoserine, phosphotyrosine, and phosphothreonine. The first epitope is different from the second epitope, so that binding of the first antibody to the protein does not interfere with the binding of the second antibody to the phosphorylated amino acid residue.

The compositions and methods described herein provide an immunoassay approach for the specific quantification of protein phosphoforms that is applicable to both manual and automated immunoassay platforms. The development and performance characteristics of novel enzyme-linked immunosorbent assays (ELISAs) for specific quantification of phosphorylated variants of proteins are described. The features of the assays described herein have been demonstrated by using IGFBP as the target protein with multiple phosphorylated isoforms. Thus, the compositions and methods described in the examples below involve an anti-IGFBP antibody in combination with an antibody that specifically binds to phosphorylated amino acid residues that are expressed on the surface of the said IGFBPs. Assay specificity is further demonstrated by showing no reactivity with antibodies that specifically bind to an unrelated phosphorylated residue, such as an anti-phosphotyrosine antibody, and by specificity of the captured molecule for a given IGFBP.

III. Components of the Compositions and Methods of the Invention

A. Target Proteins and Samples

An example of a target protein with phosphorylated variants that is measured using embodiments of the present invention is an IGFBP. Examples of IGFBP variants are IGFBP-1, IGFBP-3, and IGFBP-5, which contain phosphorylated serine amino acid residues. Other proteins that have phosphorylated variants or isoforms and are suitable for analysis by the methods described herein may be readily selected from among proteins known in the art, including a variety of enzymes, growth factors and transcription factors, among others.

Certain target proteins are present in ternary protein complexes (36), and as such are less accessible for binding to the anti-phosphorylated “site-specific” antibodies. Such target proteins are also able to be measured using the compositions and methods described herein. However, such targets are subject to additional methods steps to permit changes in the protein structure to permit binding by the antibodies, e.g., inducing a change in the ternary structure. Such changes include, without limitation, conventional treatment to permit exposure of the epitopes for binding by the first and/or second antibodies.

In various embodiments, a sample in which a phosphorylated protein concentration is measured is a biological fluid in which the protein naturally occurs. An example of a useful biological fluid that contains phophorylated proteins includes a serum sample, such as a human serum sample. Examples of human serum samples include non-pregnant serum, pregnancy serum from the first, second or third trimester. Still another suitable biological sample is amniotic fluid. Still other biological fluids that contain phosphorylated proteins suitable for the assays described herein may be selected from among known fluids, including without limitation, whole blood, plasma, urine, saliva, tears, cerebrospinal fluid, among others. Other samples may include non-naturally occurring or synthetic fluids or solutions containing phosphorylated isoforms of proteins.

B. Antibodies

Antibodies useful in the various embodiments of the compositions and methods described herein include commercially available antibodies and antibody fragments, as well as any novel antibodies generated to bind a suitable epitope on the designated target protein. The antibodies used in various embodiments exemplified herein are monoclonal or polyclonal in nature. Other antibodies and antibody fragments, such as recombinant antibodies, chimeric antibodies, humanized antibodies, antibody fragments such as Fab or Fv fragments, as well as fragments selected by screening phage display libraries, and the like are also useful in the compositions and methods described herein.

Methods for preparation of monoclonal as well as polyclonal antibodies are now well established (Harlow E. et al., 1988. Antibodies. New York: Cold Spring Harbour Laboratory). In one embodiment, antibodies are raised against recombinant human IGFBPs, synthetic fragments thereof, or IGFBP/IGF protein complexes, such as may be purified from human sera. Polyclonal antibodies are raised in various species including but not limited to mouse, rat, rabbit, goat, sheep, donkey and horse, using standard immunization and bleeding procedures. Animal bleeds with high titres are fractionated by routine selective salt-out procedures, such as precipitation with ammonium sulfate and specific immunoglobulin fractions being separated by successive affinity chromatography on Protein-A-Sepharose and leptin-Sepharose columns, according to standard methods. The purified polyclonal as well as monoclonal antibodies are then characterised for specificity and lack of cross-reactivity with related molecules. Such characterization is performed by standard methods using proteins, for example IGFBPs, labeled with a tracer such as a radioisotope or biotin in competition with increasing levels of unlabeled potential cross-reactants for antibody binding. In some embodiments, further purification is required to obtain highly specific antibody fractions or for selection of higher affinity antibody fractions from a polyclonal pool. In the case of monoclonal antibodies, care is taken to select antibodies with good binding characteristics and specificity not only for the immunogen, but also for the native circulating molecules, particularly when a recombinant molecule or peptide antigen is used for immunization. Cross-reactivity studies are further evaluated by other standard methods such as the well-established sodium dodecyl sulphate-polyacrylamide gel electrophoresis (SDS-PAGE) and Western immunoblot methods under reducing and non-reducing conditions. Evaluation of protein immunoreactivity detected in serum samples fractionated by high performance liquid chromatography (HPLC) is also used to roughly define the molecular weight profile of the protein detected (32, 37).

Monoclonal antibodies are prepared according to well established standard laboratory procedures (“Practice and Theory of Enzyme Immunoassays” by P. Tijssen (In Laboratory Techniques in Biochemistry and Molecular Biology, Eds: R. H. Burdon and P. H. van Kinppenberg; Elsevier Publishers Biomedical Division, 1985)), which are based on the original technique of Kohler and Milstein (Kohler G., Milstein C. Nature 256:495, 1975). This technique is performed by removing spleen cells from immunized animals and immortalizing the antibody producing cells by fusion with myeloma cells or by Epstein-Barr virus transformation, and then screening for clones expressing the desired antibody, although other techniques known in the art are also used. Antibodies are also produced by other approaches known to those skilled in the art, including but not limited to immunization with specific DNA.

For use in the immunoassays described herein, antibodies are purified using standard antibody purification schemes. In various embodiments, both monoclonal and polyclonal antibodies are purified by affinity chromatography over Protein-A columns. Alternatively, the antibodies are purified by affinity chromatography over a gel column containing immobilized antigen protein using standard methods.

In some embodiments of the invention, the choice of the detection antibody is based on the reported knowledge regarding the phosphorylation site(s) of the particular target protein, for example, an IGFBP. In one embodiment, as IGFBPs are mostly serine-phosphorylated (4, 23), the emphasis is on identifying strong pair-wise binding of such an antibody and a given anti-IGFBP capture antibody. If such information is not available, one of skill in the art may use antibodies with specificity for a different phosphorylated moiety (phosphoserine, phosphotyrosine, phosphothreonine).

Another consideration for selection of the appropriate antibody for use in the compositions and methods described herein is the ability of the capture antibody and the detection antibody to bind simultaneously to a given protein molecule. In one embodiment involving an IGFBP, the anti-IGFBP binding site of the capture antibody (epitope) is different from the phosphorylation site to which the detection antibody binds, thus allowing for simultaneous binding of the capture and detection antibodies and detection of the phosphorylated isoforms of the protein. In the case of significant overlap of epitopes and a resulting poor binding response, it is within the skill of one in the art to select a different anti-IGFBP antibody as the capture antibody. In some embodiments an antibody binding site is not entirely available on the surface of the protein, for example where the protein is mainly present in the sample in a complex with one or more other proteins, and is less accessible for binding to the capture or anti-phosphorylated “site-specific” antibodies. In such a circumstance, techniques known in the art are used to expose the antibody binding sites, such as partial protein denaturation or buffer modification.

As known in the art, the capture antibody is coupled with or linked to various solid phase supports using standard non-covalent or covalent binding methods, depending on the required analytical and/or solid-phase separation requirements. The solid-support is in the form of test tubes, beads, microparticles, filter paper, membranes, glass filters, magnetic particles, glass or silicon chips or other materials and approaches known to those skilled in the art. The use of microparticles, particularly magnetizable particles, that have been directly coated with the antibody (magnetic particles-capture antibody) or particles that have been labelled with a universal binder (e.g., avidin or anti-species antibody) is useful for significantly shortening the assay incubation time. These along with other alternative approaches known in the art allow for assay completion within minutes without limiting the required sensitivity. The use of magnetizable particles or similar approaches allow for convenient automation of the technology on the widely available immunoanalyzers.

The detection antibody used for detection of the phosphorylated moiety is either directly coupled with a reporter molecule, or detected indirectly by a secondary detection system. The latter is based on several different principles known in the art, including antibody recognition by a labelled anti-species antibody and other forms of immunological or non-immunological bridging and signal amplification detection systems (e.g., the biotin-streptavidin technology). The signal amplification approach is used to significantly increase the assay sensitivity and low level reproducibility and performance. The label used for direct or indirect antibody coupling is any detectable reporter molecule. Examples of suitable labels are those widely used in the field of immunological and non-immunological detection systems, such as fluorophores, luminescent labels, metal complexes and radioactive labels, as well as moieties that could be detected by other suitable reagents such as enzymes, or various combinations of direct or indirect labels such as enzymes with luminogenic substrates.

C. Buffers

The standard immunoassay matrix is a buffer-based solution containing a carrier protein (e.g., 0.05 mol/L Tris, pH 7.4, 9 g/L NaCl, 5 g/L BSA, 0.1 g/L Proclin 300) or a human or animal serum including but not limited to normal goat serum (NGS), normal equine serum (NES), or new born calf serum (NBCS). Other standard matrix preparations known in the art are also useful. One of skill in the art may readily select a buffer for various embodiments, such as those based on Tris, Borate, Phosphate or Carbonate capable of broad pH ranges.

IV. Embodiments of the Methods and Compositions of the Invention

Various embodiments of compositions and methods to measure the phosphorylated forms of IGFBPs using an immunoassay approach are described herein. The immunoassay is based on a design in which the IGFBP is captured by an anti-IGFBP antibody, and the corresponding phosphoforms and/or changes in the level of phosphorylation of the said IGFBP in a sample are then measured using an antibody against a phosphorylated residue expressed on the molecule.

In various embodiments of the methods of the invention, any sample and antibody volumes and incubation times are within the skill of one in the art to alter. These methods and compositions include common modifications used in conventional immunoassays, and any modification known to those skilled in the art. In various embodiments, the assay design is homogeneous or heterogeneous, depending on the particular application of the assay and the need for speed, sensitivity, accuracy and convenience.

Various embodiments allow the accurate tracking of changes in the state of protein phosphorylation in response to changes in pathophysiological conditions of interest. The specific quantification and monitoring of changes in the level of protein phosphorylation are more informative in relation to measuring the total protein immunoreactivity than the currently available immunoassays, for example, for IGFBP-1 (32) or IGFBP-3 (37). Relating the concentrations of phosphorylated protein isoforms to the total concentrations of the protein, such as by using a “ratio” determination, is useful when assessing pathophysiological conditions in which changes in the phosphorylation levels of the protein are greater than changes in its total immunoreactivity levels. Availability of such immunoassays and methods for their use facilitate investigations of the pathophysiological roles and potential diagnostic values of phosphorylated proteins in general and of IGFBPs in particular.

The following examples are provided for the purpose of illustrating various embodiments of the invention and are not meant to limit the present invention in any fashion:

### EXAMPLE 1

### Phosphorylated IGFBP Immunoassay

Sample Preparation.

Serum samples from non-pregnant females (n=29, age 17-48, median), and from first (n=38) and second (n=29) trimester pregnancies were obtained from Lenetix Medical Screening Laboratory Inc., (New York, N.Y.). These specimens were residuals from routine or research test samples. After collection, blood samples were allowed to clot and were then separated. After clinical testing, the residuals were stored at −20° C. and used for the present studies within 3 months. Amniotic fluids (n=20) from second trimester pregnancies (15-18 week gestations) were obtained form clinical laboratories in Toronto, ON, Canada. The samples were residuals from routine clinical test samples and were stored at −70° C. for fewer than 4 months before use.

Reagents

Horseradish peroxidase (HRP) was obtained from Scripps Labs., San Diego, Calif. Tetramethylbenzidine (TMB) microwell peroxidase substrate system was from Neogn Corporation, Lexington, Ky. Sulfosuccinimidyl 4-(N-maleimidomethyl) cyclohexane-1-carboxylate (sulfo-SMCC) and 2-iminothiolane were purchased from Pierce, Rockford, Ill. Enzyme immunoassay-grade alkaline phosphatase (ALP) was obtained from Boehringer Mannheim, Indianapolis, Ind. All other chemical reagents were of highest quality and were obtained from Sigma Chemical, St. Louis, Mo. or Amresco, Solon, Ohio. Microtitration strips and frames were products of Greiner International, Germany.

Recombinant human IGF-I and IGF-II were obtained from GroPep, Adelaide, Australia and recombinant nonglycosylated human IGFBP-3 from Celtrix Pharmaceuticals, Santa Clara, Calif. Recombinant human IGFBP-2 and IGFBP-4, IGFBP-5 and IGFBP-6 were purchased from Austral Biologicals, San Roman, Calif. Human IGFBP-I, purified from human amniotic fluid according to previously described methods (35), was obtained from Diagnostic Systems Laboratories, Inc. (Webster, Tex.). The preparation was calibrated against pure recombinant human IGFBP-1. Recombinant human IGFBP-5 expressed in a mouse myeloma cell line was purchased from R&D systems, Minneapolis, Minn.

Antibodies.

Mouse monoclonal antibodies against the various phosphorylated amino acid residues were purchased from commercial sources. Anti-phosphoserine antibody was obtained from BD Biosciences, San Jose; Calif. Anti-phosphotyrosine was obtained from Upstate laboratories, Lake Placid, N.Y. Five different IGFBP-1 mouse monoclonal antibodies and an affinity-purified goat polyclonal anti-IGFBP-1 antibody were obtained from Diagnostic Systems Laboratories, Inc. (Webster, Tex.). The specificity of these antibodies for IGFBP-1 has been described previously (reference 32, U.S. Pat. No. 5,747,273). Among these antibodies, a monoclonal anti-IGFBP-1 antibody that was unaffected by the state of IGFBP-1 phosphorylation or IGFBP-1 binding to IGF-I was previously used in designing an immunoassay for Total IGFBP-1 (reference 32, U.S. Pat. No. 5,747,273). Because of these characteristics, the same antibody was identified as a candidate antibody and evaluated for development of the present ELISA for specific measurement of the phosphorylated IGFBP-1 variants. A panel of anti-IGFBP-5 monoclonal (n=12) and polyclonal (n=4) antibodies raised against recombinant human IGFBP-5 or a synthetic peptide of IGFBP-5 were also obtained from Diagnostic Systems Laboratories, Inc. (Webster, Tex.).

As reported previously, a monoclonal and a polyclonal antibody against intact IGFBP-5 generated the highest binding signal and specificity when evaluated in pair-wise ELISA combinations or in a non-competitive enzyme immunoassay (EIA) format (33, 34). Because of this characteristic and the finding that the polyclonal coating was less affected by IGF-I binding to IGFBP-5, both of these antibodies were evaluated in more detail. The anti-IGFBP-3 antibody evaluated was a monoclonal antibody that was previously shown to bind to the N-terminal region of IGFBP-5 and was instrumental in developing an ELISA methodology for measuring ternary IGFBP-3/IGF-I complexes (reference 26, U.S. Pat. No. 6,248,546). All mouse monoclonal antibodies and goat polyclonal antibodies to IGFBPs were obtained from Diagnostic Systems Laboratories, Inc. (Webster, Tex.). These antibodies with acceptable specificity and binding characteristics have been incorporated into specific immunoassays for IGFBP-1 (32), IGFBP-3 (37), or IGFBP-5 (33, 34).

Reagent Preparation Protocols.

Antibody was coated to microwells (250-1000 ng/100 μL/well) according to protocols previously described (38-40). Antibody conjugation to biotin or HRP was conducted as previously described (38-40). Standards were prepared by appropriately diluting recombinant human IGFBP or a human serum pool into various standard matrix buffers to produce the desired IGFBP standards in arbitrary units. The serum pool was assigned values in mass units based on the concentration of the pool measured by a conventional immunoassay for the corresponding total IGFBP immunoreactivity. For example, for calibration of the phosphorylated IGFBP-1 ELISA, a serum pool made by mixing up to 10 different samples with high phosphorylated IGFBP-1 content was assayed for total IGFBP-1 immunoreactivity by the Diagnostic Systems Laboratories, Inc. Total IGFBP-1 ELISA (32). The obtained value was then assigned to the serum pool and used for standard preparation. The standard matrix used for both phosphorylated IGFBP-1 and IGFBP-5 ELISAs was a commercially prepared new born calf serum For both phosphorylated IGFBP-1 and IGFBP-5 ELISAs, a Tris-based assay buffer (0.025 M Tris-HCl, pH 6.1, containing 0.5 g FSG, 0.5 g Brij 35 and 2 mL procline 300 per litre) was selected.

The anti-IGFBP antibodies were purified using standard antibody purification schemes. Both monoclonal and polyclonal antibodies were purified by affinity chromatography over Protein-A columns by affinity chromatography over a gel column containing immobilized IGFBP using standard methods.

Assay Protocols

In one embodiment, a one-step assay (simultaneous incubation of sample plus detection antibody) was performed; in another embodiment, a two-step assay (sequential incubation of sample and the detection antibody) was performed.

IGFBP antibody evaluation in pair-wise combinations with commercially available anti-phosphoserine or anti-phosphotyrosine (negative control) antibodies were conducted using conventional methods. Conditions affording a reasonable response were selected and evaluated further.

The phosphorylated IGFBP-1 and IGFBP-5 ELISAs involved the steps of addition of standards, samples or controls (0.02-0.05 mL) and the assay buffer (0.05-0.10 mL) in duplicate to the anti-IGFBP antibody pre-coated wells, followed by 1-2 hr incubation at room temperature with continuous shaking. The wells were then washed five times and incubated for 30-60 min as above with 0.10 mL/well of the detection anti-phosphoserine antibody. After an additional washing step, the wells were incubated with 0.1 mL/well TMB/H₂O₂ substrate solution and incubated for an additional 10-min as above. Stopping solution (0.1 mL) was then added and absorbance was measured by dual wavelength measurement at 450 nm with background wavelength correction set at 620 nm. ELISA absorbance measurements were performed with the Labsystems Multiskan Multisoft microplate reader (Labsystems, Helsinki, Finland). The composition of the coating and blocking buffers and the antibody coating procedure to microtitration wells as well as the wash and stopping solutions were as described previously (32, 39).

Coupling of the detection antibodies to HRP was performed as described (32, 39). The coupling reaction involved activation of the enzyme with sulfo-SMCC and its subsequent conjugation to the detection antibody, which had been activated by 2-iminothiolane. The stock HRP-conjugated antibody solution was diluted at least 1000-fold prior to use.

For both phosphorylated IGFBP-1 and IGFBP-5 assays, standards were pre-selected serum pools with high endogenous phosphorylated IGFBP-1 or IGFBP-5 content. The IGFBP-1 pool was assayed for total IGFBP-1 immunoreactivity by the Diagnostic Systems Laboratories, Inc. Total IGFBP-1 ELISA (32) at various dilutions, and the mean value was assigned to the serum pool and then diluted in the standard matrix to give standard values of 0, 1.56, 3.13, 6.25, 12.5, 25, 50, and 100 ng phosphorylated IGFBP-1/mL. A similar approach was used to calibrate the phosphorylated IGFBP-5 ELISA. However, because of unavailability of a commercial assay for IGFBP-5, the pool was assigned a value of 100 units/mL and then serially diluted in the standard matrix to concentrations of 0, 1.56, 3.13, 6.25, 12.5, 25, 50, and 100 units phosphorylated IGFBP-5/mL. The standards were stable for at least 2 days at 4° C. The quality control samples used were fresh serum samples containing various levels of phosphorylated IGFBPs. The nominal concentrations of the control samples were established by analyzing the samples in a total IGFBP ELISA (Diagnostic Systems Laboratories, Inc.).

Phosphorylated IGFBP ELISA Validation Procedures.

The lower limit of detection (sensitivity) was determined by interpolating the mean plus two standard deviations (2SD) of 12 replicate measurements of the zero calibrator (NBCS). The intra-assay coefficients of variability (CVs) were determined by replicate analysis (n=12) of four samples at different concentrations in one run and inter-assay CVs by duplicate measurement of samples in 9-12 separate assays. Recovery was assessed by adding 25 μL of high concentration IGFBP sample to 225 μL of three low concentration IGFBP samples and analyzing the supplemented and un-supplemented samples. Percent recovery was determined by comparing the amount of added IGFBP with the amount measured after subtracting the endogenous IGFBP concentrations. Linearity was tested by analyzing three serum samples serially diluted (2- to 16-fold) in the zero calibrator of the assay.

Other Assays.

Total IGFBP-1 and non-phosphorylated IGFBP-1 were assayed as previously reported (32), using Diagnostic Systems Laboratories, Inc. Total and Non-phosphorylated IGFBP-1 ELISA immunoassays.

Dephosphorylation of IGFBPs.

Dephosphorylation of IGFBP-1 was achieved by sample pretreatment with alkaline phosphatase (ALP) using a similar procedure described previously (32). Briefly, ALP dissolved in 10 μL of 1 mol/L diethanolamine (DEA), pH 9.5, containing 0.5 mmol/L MgCl₂ was added to a 200-μL aliquot of the sample, mixed, and incubated at room temperature for 2 hours. The untreated control aliquot received 10 μL of the DEA buffer and was similarly incubated. ALP-treated and untreated samples were then analyzed.

Data Analysis.

ELISA data were analyzed with a data reduction software package included with the instrumentation, using a cubic spline (smoothed) curve fit. Descriptive data are presented as the mean, median, and standard deviation unless otherwise specified. Linear regression analysis was performed by the least-squares method, and correlation coefficients were determined by the Pearson method. The plotting and statistical analysis were performed using SigmaPlot and SigmaStat software (Systat Software Inc, Point Richmond, Calif. 94804-2028).

Phosphorylated IGFBP ELISA.

Assessment of panels of well characterized IGFBP-1, IGFBP-3, and IGFBP-5 monoclonal and polyclonal antibodies in pair-wise combination with antibodies recognizing phosphorylated amino acid residues (e.g., phosphoserine, phosphotyrosine) provided information on their binding characteristics, particularly identifying antibody combinations capable of specific detection of phosphoforms of the various IGFBPs. Phosphoforms of IGFBP-1 and IGFBP-5 were detectable. Immunoassay format detection of phosphoforms of IGFBP-3 needed further amplification of the signal generated and/or additional treatment to change the circulation structure of IGFBP-3 prior to assay. Blood IGFBP-3 is mainly present in ternary protein complexes (36), and is less accessible for binding to the anti-phosphorylated “site-specific” antibodies.

As per conventional immunoassays, pair-wise antibody selection was based on their relative binding responses in relation to non-specific binding signal (NSB) generated by the zero-dose standard (signal-to-noise ratios). IGFBPs were captured by a monoclonal antibody followed by selective detection of the captured phosphorylated subtypes by an antibody that specifically detects the exposed phosphoserine residue.

Useful analytical performance characteristics were obtained with a coating antibody concentration of 5 mg/L (500 ng/0.1 mL per well), a detection antibody concentration of about 0.1-0.25 mg/L (10-25 ng/0.1 mL per well), a sample size of 0.025-0.05 mL, a first- and second-step room temperature incubation of 2 hours and 1 hour, respectively, and a 10-min substrate development step.

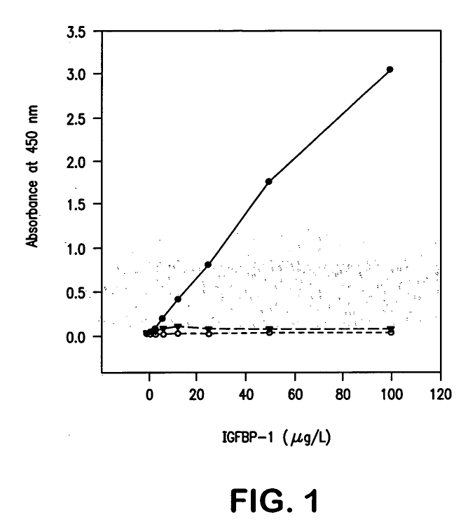

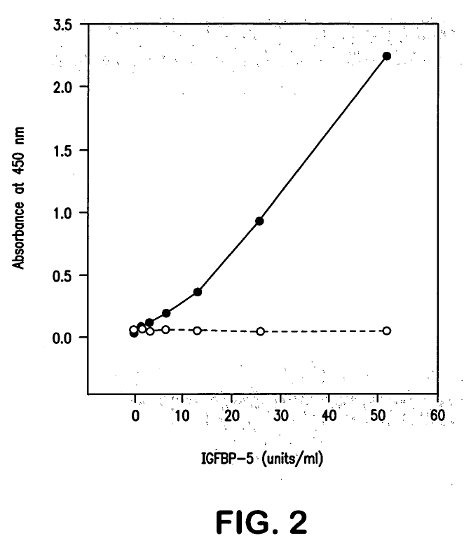

Standard curve results for the Phosphorylated IGFBP-1 ELISA and the Phosphorylated IGFBP-5 ELISA are shown in FIGS. 1 and 2, respectively. As a representative example, the analytical performance characteristics of the Phosphorylated IGFBP-1 ELISA were assessed in more detail using conventional approaches. The relevant performance data are summarized in Table 1.

TABLE 1
Phosphorylated IGFBP-1 ELISA Validation Data
Assay parameter

| | |
|---|---|
| Detection limit (ng/mL) | 0.30 |
| Standard range (ng/ml) | 1.56-100 |
| Intraassay CV, % | 2.1-8.6 |
| Interassay CV, % | 4.0-7.3 |
| Recovery of additions, % | 97.8 ± 9.2 |
| Recovery after dilution, % | 93.4 ± 6.0 |

Specificity

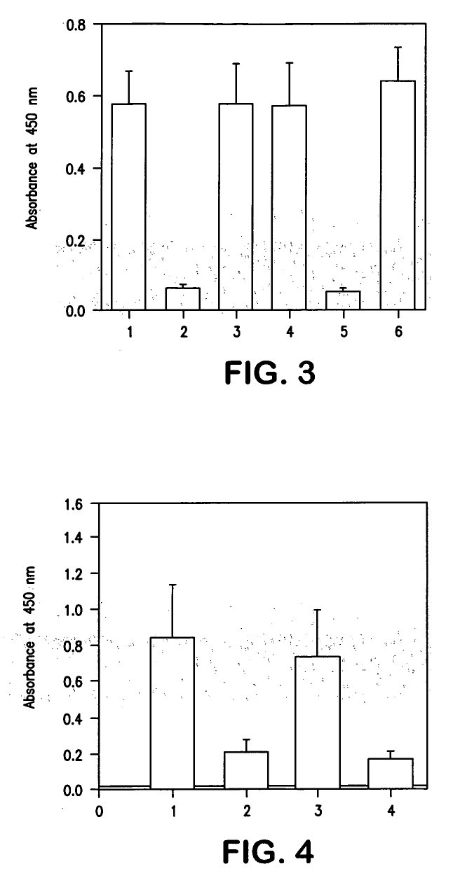

IGFBP antibodies herein were previously shown to have minimal or no cross-reactivity with other members of the IGFBP family (32-34, 37). To demonstrate protein phosphoform specificity, the binding response of the assays to an anti-phosphotyrosine antibody as well as to the anti-phosphoserine detection antibody, before and after sample dephosphorylation by alkaline phosphatase, was assessed. Dephosphorylation of IGFBP in serum by exogenously added alkaline phosphatase was performed as previously described (32-34). As expected and as shown in FIG. 1, there was no significant binding of the anti-phosphotyrosine antibody to the captured IGFBP-1 phosphoforms. A similar observation was made when IGFBP-5 captured by a polyclonal or by one of three different monoclonal antibodies was tested against the anti-phosphotyrosine antibody (FIGS. 2 and 3). In both cases, the expected binding of the anti-phosphoserine detection antibody was nearly eliminated in response to IGFBP-1 or IGFBP-5 dephosphorylation by alkaline phosphatase (FIGS. 1 and 4).

### EXAMPLE 2

### Physiological Investigations

Phosphorylated IGFBP-1 in Physiological Fluids.

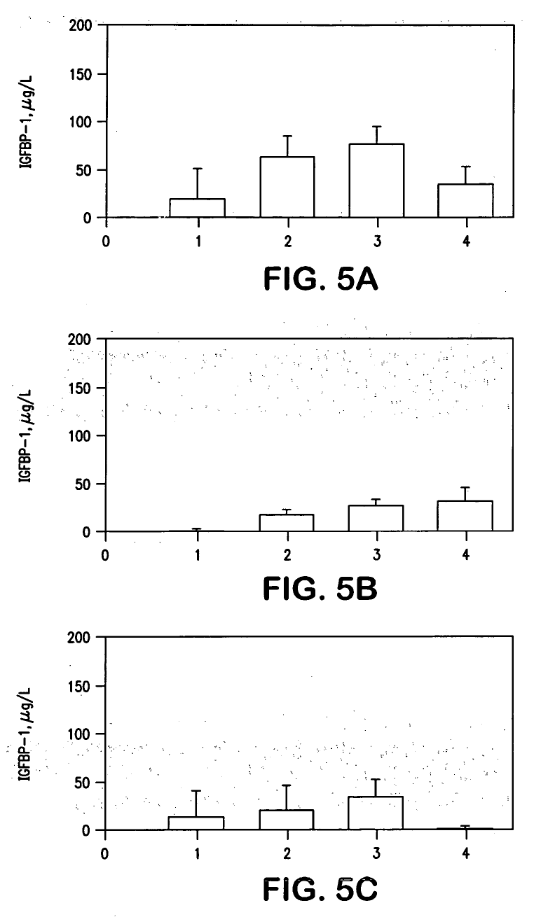

Phosphorylated IGFBP-1 was measured in non-pregnant adult serum samples, in first and second trimester pregnancy sera, and in amniotic fluid. The Phosphorylated IGFBP-1 ELISA measured significantly different concentrations in the various sample types, with the highest levels detectable in the second trimester samples and the lowest levels in amniotic fluid (See FIGS. 5A-5C).

Non-Pregnancy Samples.

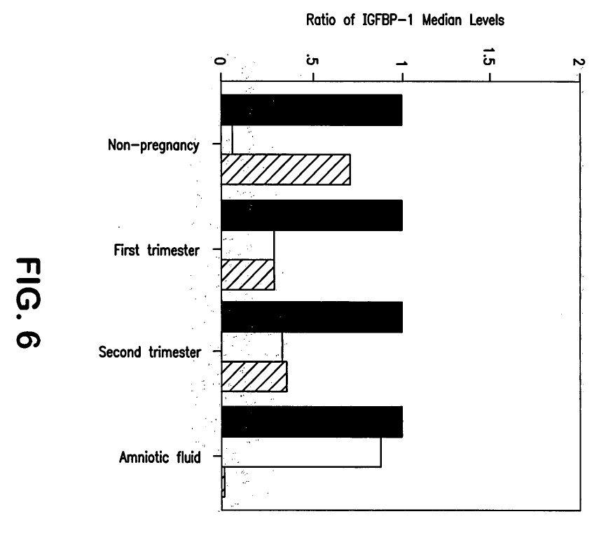

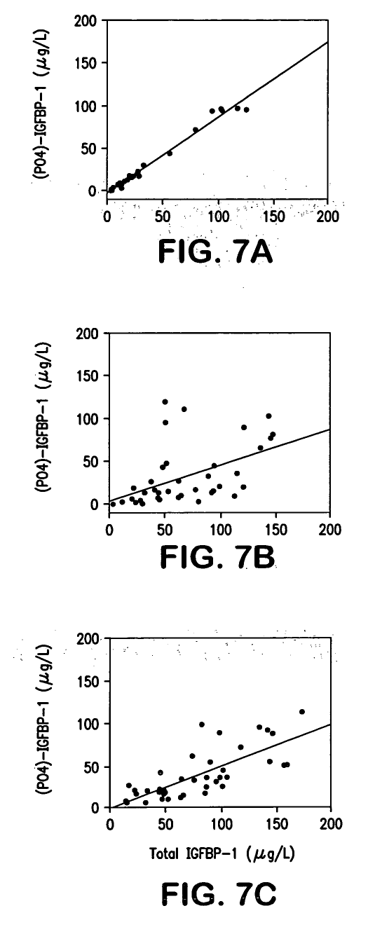

In the randomly selected non-pregnancy sera, the phosphorylated and the total IGFBP-1 median levels were measured using an immunoassay as described herein, and the Diagnostic Systems Laboratories, Inc. Total IGFBP-1 ELISA, respectively. These two levels were highly similar (p=0.225 by ANOVA) (FIGS. 5A-5C and 6), and the individual values were highly correlated (FIGS. 7A-7C). The highly similar concentrations between the total IGFBP-1 and the phosphorylated IGFBP-1 variants were not unexpected, as IGFBP-1 in normal adult sera is predominantly phosphorylated (20, 32). The latter was consistent with the detection by the Diagnostic Systems Laboratories, Inc. non-phosphorylated IGFBP-1 ELISA of significantly different (p=<0.001) and much lower levels of non-phosphorylated IGFBP-1 relative to the phosphorylated or total levels (FIGS. 5A-5C).

Pregnancy Samples.

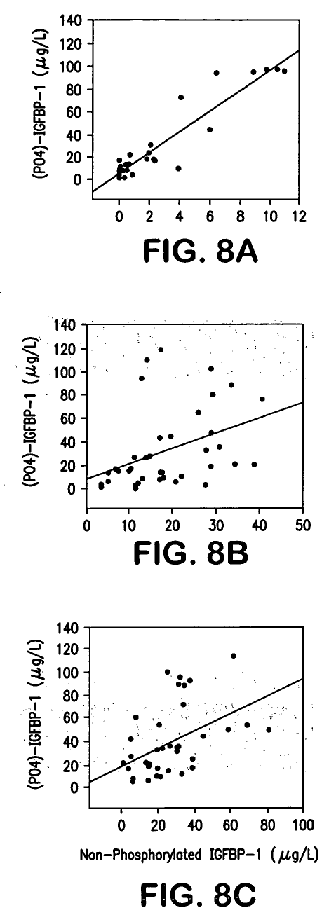

In both first and second trimester samples, the median phosphorylated IGFBP-1 levels were found to be significantly lower than the corresponding total IGFBP-1 levels (p=<0.001) measured by the Diagnostic Systems Laboratories, Inc. Total IGFBP-1 ELISA (FIG. 6). This was in contrast to statistically similar medians for the phosphorylated versus non-phosphorylated levels of IGFBP-1 detected in the first (p=0.09) and second (p=0.152) trimester samples, respectively. Changes in the level of IGFBP-1 phosphorylation in association with pregnancy showed variability between individual samples, in comparisons of the phosphorylated versus total (FIGS. 7B-7C) or phosphorylated versus the non-phosphorylated (FIGS. 8B-8C) IGFBP-1 levels in first and second trimester samples.

Amniotic Fluid.

Amniotic fluid contains predominantly non-phosphorylated IGFBP-1 (20). Accordingly, the Phosphorylated IGFBP-1 ELISA measured comparatively low levels of IGFBP-1 immunoreactivity in the amniotic fluid samples, while the Diagnostic Systems Laboratories, Inc. Total and Non-phosphorylated IGFBP-1 ELISAs detected relatively similar and significantly higher concentrations (FIGS. 5A-5C and 6).

The following references are cited herein:
1. Rotwein P. Structure, evolution, expression and regulation of insulin-like growth factors I and II [Review]. Growth Factors 1991; 5:3-18.
2. Daughaday W H, Rotwein P. Insulin-like growth factors I and II. Peptide, messenger ribonucleic acid and gene structures, serum and tissue concentration. Endocr Rev 1989; 10:68-91.
3. LeRoith D, Adamo M, Werner H, Roberts C T, Jr. Insulin-like growth factors and their receptors as growth regulators in normal physiology and pathological states [Review]. Trends Endocrinol Metab 1991; 2:134-139.
4. Jones J I, Clemmons D R. Insulin-like growth factors and their binding proteins: biological actions. Endocr Rev 1995; 16:3-34.
5. Cohen P, Rosenfeld R G. Physiologic and clinical relevance of the insulin-like growth factor binding proteins [Review]. Curr Opin Pediatr 1994; 6:462-467.
6. Rajarm S., Baylink D J, Mohan S. Insulin-like growth factor-binding proteins in serum and other biological fluids: regulation and functions. Endocrine Rev 1997; 18:801-831.
7. Koistinen R, Kalkkinen N, Huhtala M L, Seppala M, Bohn H, Rutanen E-M. Placental protein 12 is a decidual protein that binds somatomedin and has an identical N-terminal amino acid sequence with somatomedin binding protein from human amniotic fluid. Endocrinology 1986; 118:1375-1378.
8. Bell S C, Keyte J W. N-terminal amino acid sequence of human pregnancy-associated endometrial alpha 1-globulin, an endometrial insulin-like growth factor (IGF) binding protein: evidence for two small molecular weight IGF binding proteins. Endocrinology 1988; 123:1202-1204.
9. Lee P D K, Conover C A, Powell D R. Regulation and function of insulin-like growth factor binding protein-1. Proc Soc Exp Biol Med 1993; 204:4-29.
10. Suikkari A-M, Jalkanen J, Koistinen R, Butzow R, Ritvos O, Ranta T, Seppala M. Human granulosa cells synthesize low molecular weight insulin-like growth factor-binding protein. Endocrinology 1989; 124:1088-1090.
11. Giudice L C. Insulin-like growth factors and ovarian follicular development. Endocr Rev 1992; 13:641-669.
12. Rutanen E-M, Bohn H, Seppala M. Radioimmunoassay of placental protein 12: levels in amniotic fluid, cord blood, and serum of healthy adults, pregnant women, and patients with trophoblastic disease. Am J Obstet Gynecol 1982; 144:460-463.
13. Hills F A, English J, Chard T. Circulating levels of IGF-I and IGF-binding protein-I throughout pregnancy: relation to birth weight and maternal weight. J Endocrinol 1996; 148:303-309.
14. Verhaeghe J, Billen J, Giudice L C. Insulin-like growth factor-binding protein-1 in umbilical artery and vein of term fetuses with signs suggestive of distress during labor. J Endocrinol 2001; 170: 585-590.
15. Kajantie E. Insulin-like growth factor (IGF)-I, IGF binding protein (IGFBP)-3, phosphoisoforms of IGFBP-1 and postnatal growth in very-low-birth-weight infants. Horm Res 2003; 60: 124-130.
16. Sakai K, D'Ercole A J, Murphy L J, Clemmons D R. Physiological differences in insulin-like growth factor binding protein-I phosphorylation in IGFBP-1 transgenic mice. Diabetes 2001; 50:32-38.
17. Watson C, Bialek P, Khosravi J, Yee Siu-Pok, Han V. Elevated circulating insulin-like growth factor binding protein-1 is sufficient to cause fetal growth restriction. 2005 (in preparation).
18. Jones J I, D'Ercole A J, Camacho-Hubner C, Clemmons D R. Phosphorylation of insulin-like growth factor (IGF)-binding protein 1 in cell culture and in vivo: effects on affinity for IGF-I. Proc Natl Acad Sci USA 1991; 88:7481-7485.
19. Koistinen R, Angervo M, Leinonen P, Hakala T, Seppala M. Phosphorylation of insulin-like growth factor-binding protein-1 increases in human amniotic fluid and decidua from early to late pregnancy. Clin Chem Acta 1993; 215:189-199.
20. Westwood M, Gibson J M, Davies A J, Young R J, White A. The phosphorylation pattern of insulin-like growth factor-binding protein-1 in normal plasma is different from that in amniotic fluid and changes during pregnancy. J Clin Endocrinol Metab 1994; 79:1735-1741.
21. Graves J D, Krebs E G. Protein phosphorylation and signal transduction. Pharmacol Ther 1999; 82:111-121.
22. Garcia B A, Shabanowitz J, Hunt D F. Analysis of protein phosphorylation by mass spectrometry. Methods 2005; 35:256-264.
23. Kelly K M, Oh Y, Gargosky S E, Gucev Z, Matsumoto T, Haw V, Ng L, Simpson D M, Rosenfeld R G. Insulin-like growth factor binding proteins (IGFBPs) and their regulatory dynamics. Int J Biochem Cell Biol 1996; 28:619-637.
24. Westwood M, Gibson J M, Williams A C, Clayton P E, Hamberg O, Flyvbjerg A, White A. Hormonal regulation of circulating IGFBP-1 phosphorylation Status. J Clin Endocrinol Metab 1995; 80:3520-3527.
25. Bankowski E, Sobolewski K, Palka J, Jawoski S. Decreased expression of the insulin-like growth factor-I-binding protein-1 (IGFBP-1) phosphoisoform in pre-eclampsic Warton's jelly and its role in the regulation of collagen biosynthesis. Clin Chem Lab Med 2004; 42:175-181.
26. Heald A H, Siddals K W, Fraser W, Taylor W, Kaushal K, Morris J, Young R J, White A, Gibson J M. Low circulating levels of insulin-like growth factor binding protein-1 (IGFBP-1) are closely associated with the presence of marcrovascular disease and hypertension in type 2 diabetes. Diabetes 2002; 18:2629-2636.
27. Schedlich L J, Nilsen T, John A P, Jans D A, Baxter R C. Phosphorylation of insulin-like growth factor binding protein-3 by deoxyribonucleic acid-dependent protein kinase reduces ligand binding and enhances nuclear accumulation. Endocrinol 2003; 144:1984-1993.
28. Mishra S, Murphy L J. Phosphorylation of insulin-like growth factor (IGF) binding protein-3 by breast cancer cell membranes enhances IGF-I binding. Endocrinol 2003; 44:4042-4050.
29. Hou J, Clemmons D R, Smeekens S. Expression and characterization of serine protease that preferentially cleaves insulin-like growth factor binding protein-5. J Cell Biochem 2005; 94:470-484.
30. Lin K, Rath V L, Dai S C, Fletterick R J, Hwang P K. A protein phosphorylation switch at the conserved allosteric site in GP. Science 1996; 273:1539-1542.
31. Rott I. The primary interaction with antigen in “essential immunology” Blackwell scientific publications, Boston Mass., 1994; 8^{th} edition: 81-102.
32. Khosravi M J, Diamandi A, Mistry J. Immunoassay of insulin-like growth factor binding protein-1. Clin Chem 1997; 43:523-532.
33. Khosravi M J, Diamandi A, Mistry J, Bandla M, Krishna R G. Development, evaluation, and application of monoclonal and polyclonal antibodies in enzyme-linked immunosorbent assay (ELISA, EIA) of insulin-like growth factor binding protein-5. The Endocrine Society 81^{st} Annual Meeting, P2-563, 1999
34. Khosravi M J, Diamandi A, Mistry J, Bandia M, Krishna R G. Altered IGFBP-5 Immunoreactivity in response to changes in its state of phosphorylation, Mg2+ and pH: indications for phosphorylation (Mg2+)-dependent regulation of IGFBP-5 bioactivity. 5^{th} International Symposium on IGFs. Brighton, England, 1999.
35. Powell D R, Lee P D K, Shively J E, Eckenhausen M, Hintz R L. Method for purification of an insulin-like growth factor-binding protein produced by human HEP-G2 hepatoma cells. J Chromatogr B Biomed Appl 1987; 420:163-170.
36. Khosravi J, Diamandi A, Mistry J, Krishna R G. 1999 The high molecular weight insulin-like growth factor binding protein complex: Epitope mapping, immunoassay, and preliminary clinical evaluation. J Clin Endocrinol Metab 84:2826-2833.
37. Diamandi A, Mistry J, Krishna R G, Khosravi J ((SRA). Immunoassay of insulin-like growth factor binding protein-3: new means to quantifying IGFBP-3 proteolysis. J Clin Endocrinol Metab 85; 2327-2333, 2000.
38. Khosravi M J, Morton R C. Novel application of streptavidin-hapten derivative as protein-tracer conjugate in competitive-type immunoassays involving biotinylated detection probes. Clin Chem 1991; 37:58-63.
39. Khosravi M J, Papanastasiou-Diamandi A, Mistry J. Ultrasensitive immunoassay for prostate-specific antigen based on conventional colorimetric detection. Clin Biochem 1995; 28:407-414.
40. Khosravi M J, Diamandis E P. Immunofluorometry of choriogonadotropin by time-resolved fluorescence spectroscopy, with a new europium chelate as label. Clin Chem 1987; 33:1994-1999.
41. Khosravi M J, Diamandi A, Mistry J, Lee P D K. Noncompetitive ELISA for human serum insulin-like growth factor-I. Clin Chem 1996; 42:1147-1154.
42. Khosravi M J, Diamandi A, Mistry J, Krishna R G, Khare A. Acid-labile subunit of human insulin-like growth factor binding protein complex: measurement, molecular, and clinical evaluation. J. Clin Endocrinol Metab 1997; 82:3944-3951.
43. Khosravi J, Diamandi A, Mistry J, Scorilas A. Insulin-like growth factor (IGF)-I and IGF binding protein (IGFBP)-3 in benign prostatic hyperplasia and prostate cancer. J Clin Endocrinol Metab 2001; 86:694-699.
44. Stultz C M, Levin A D, Edelman E R. Phosphorylation-induced conformational changes in a mitogen-activated protein kinase substrate, J Biol Chem 2002; 277:47653-47661.
45. Rosenzweig S A. What's new in the IGF-binding proteins? GH & IGF Res. 2004; 14:329-336

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the various embodiments of the inventions are well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. An immunoassay composition comprising a first antibody and a second antibody, wherein the first antibody binds to a protein having a phosphorylated amino acid residue, wherein the second antibody binds to the phosphorylated amino acid residue, and wherein the first antibody does not bind to the phosphorylated amino acid residue.

2. The composition of claim 1, wherein the protein comprises an IGFBP.

3. The composition of claim 2, wherein the IGFBP is selected from the group consisting of IGFBP-1, IGFBP-3, and IGFBP-5.

4. The composition of claim 1, wherein the phosphorylated amino acid residue is selected from the group consisting of phosphoserine, phosphotyrosine, and phosphothreonine.

5. The composition of claim 1, further comprising a solid support coupled with the first antibody.

6. The composition of claim 5, wherein the solid support comprises a protein coupling surface selected from the group consisting of a microtiter plate, a colloidal metal particle, an iron oxide particle and a polymeric bead.

7. The composition of claim 1, further comprising the second antibody coupled with a label.

8. The composition of claim 7, wherein the label comprises a chemiluminescent agent, a colorimetric agent, an energy transfer agent, an enzyme, a substrate of an enzyme reaction, a fluorescent agent or a radioisotope.

9. The composition of claim 8, wherein the enzyme is selected from the group consisting of alkaline phosphatase, amylase, luciferase, catalase, beta-galactosidase, glucose oxidase, glucose-6-phosphate dehydrogenase, hexokinase, horseradish peroxidase, lactamase, urease and malate dehydrogenase.

10. An immunoassay method for measuring a concentration of a protein having a phosphorylated amino acid residue in a sample, comprising the steps of:
(a) binding a first antibody to a protein having a phosphorylated amino acid residue, thereby creating a bound first antibody;
(b) binding a second antibody to the phosphorylated amino acid residue, thereby creating a bound second antibody;
(c) measuring an amount of the bound second antibody; and
(d) calculating the concentration of the protein in the sample based on the amount of bound second antibody.

11. The method of claim 10, wherein the protein comprises an IGFBP.

12. The method of claim 11, wherein the IGFBP is selected from the group consisting of IGFBP-1, IGFBP-3, and IGFBP-5.

13. The method of claim 12, wherein the phosphorylated amino acid residue is selected from the group consisting of phosphoserine, phosphotyrosine, and phosphothreonine.

14. The method of claim 10, further comprising a solid support coupled with the first antibody.

15. The method of claim 14, wherein the solid support comprises a protein coupling surface selected from the group consisting of a microtiter plate, a colloidal metal particle, an iron oxide particle, and a polymeric bead.

16. The method of claim 10, further comprising the second antibody coupled with a label.

17. The method of claim 16, wherein the label comprises a chemiluminescent agent, a calorimetric agent, an energy transfer agent, an enzyme, a substrate of an enzyme reaction, a fluorescent agent or a radioisotope.

18. The method of claim 17, wherein the enzyme is selected from the group consisting of alkaline phosphatase, amylase, luciferase, catalase, beta-galactosidase, glucose oxidase, glucose-6-phosphate dehydrogenase, hexokinase, horseradish peroxidase, lactamase, urease and malate dehydrogenase.

19. An immunoassay kit for measuring a concentration of a protein having a phosphorylated amino acid residue in a sample, comprising:
(a) a first antibody and a second antibody, wherein the first antibody binds to a protein having a phosphorylated amino acid residue and the second antibody binds to the phosphorylated amino acid residue;
(b) a solid support coupled with the first antibody; and
(c) a label coupled with the second antibody.

20. The composition of claim 19, wherein the protein comprises an IGFBP.

21. The composition of claim 20, wherein the IGFBP is selected from the group consisting of IGFBP-1, IGFBP-3, and IGFBP-5.

22. The composition of claim 19, wherein the phosphorylated amino acid residue is selected from the group consisting of phosphoserine, phosphotyrosine, and phosphothreonine.

23. An immunoassay method for measuring a phosphorylation level of a protein sample, comprising the steps of:
(a) contacting a first antibody with a protein sample, wherein the protein sample comprises a protein having a phosphorylated amino acid residue, and wherein the first antibody binds to the protein, thereby creating a bound first antibody;
(b) binding a second antibody to the phosphorylated amino acid residue, thereby creating a bound second antibody;
(c) measuring an amount of bound second antibody;
(d) calculating a concentration of the protein having a phosphorylated amino acid residue in the sample, based on the amount of bound second antibody;
(e) measuring a concentration of total protein in the protein sample, and
(f) relating the concentration of the protein having a phosphorylated amino acid residue to the concentration of total protein in the sample.

24. The method of claim 23, wherein relating the concentration of the protein having a phosphorylated amino acid residue to the concentration of total protein in the sample comprises calculating a ratio of the concentration of the protein having a phosphorylated amino acid residue and the concentration of total protein in the sample.

25. The method of claim 23, wherein the protein sample comprises a biological fluid.

26. The method of claim 25, wherein the biological fluid is selected from the group consisting of non-pregnant serum, pregnancy serum, and amniotic fluid.

27. The method of claim 26, wherein the pregnancy serum is selected from the group consisting of first trimester serum, second trimester serum and third trimester serum.
